(19) United States
(12) Patent Application Publication
(10) Pub. No.: US 2007/0082048 A1
(43) Pub. Date: Apr. 12, 2007

(54) Sleep aid formulations

(76) Inventor: Ronald Warner, (US)

Correspondence Address: Patrick J. Halloran, Ph.D., J.D., 3141 Muirfield Road, Center Valley, PA, 18034, US

(21) Appl. No.: 11/449,573

(22) Filed: Jun. 8, 2006

Related U.S. Application Data
(60) Provisional application No. 60/688,772, filed on Jun. 8, 2005.

(51) Int. Cl. A61K 31/5513; A61K 9/20

(52) U.S. Cl. 424/464; 514/221

(57) ABSTRACT

Orally dissolvable dosage forms comprising sleep aids such as benodiazepines are described. The dosage forms may be in the form of tablets or strips. In certain embodiments, the dosage forms may comprise particles, granules, microgranules, or crystals, and a matrix. The particles, granules or microgranules comprise a combination of immediate-release particles, granules or microgranules with sustained-release, delayed-release or enteric-coated particles, granules or microgranules. In one embodiment, the sleep aid is diazepam, triazolam, midazolam, temazepam, flurazepam, zolpidem, zaleplon or a combination thereof. The sleep aids are typically employed in pharmaceutically effective amounts to treat insomnia.

This application claims priority to Ser. No. 60/688,772 filed Jun. 8, 2005.

### BACKGROUND

Many physiological functions are characterized by diurnal rhythms, in which levels of circulating hormones, catecholamines and other compounds fluctuate during the day and/or night. Certain medical disorders, such as insomnia, are associated with abnormalities in these rhythms. The term “insomnia” refers to the perception of inadequate or non-restful sleep by a patient. The prevalence of insomnia has also been shown to be related to the age and sex of the individuals, being higher in older individuals and in females.

During the 1980's, the pharmaceutical treatment of insomnia shifted away from barbiturates and other CNS depressants toward the benzodiazepine class of sedative-hypnotic agents. This class of compounds produces a calming effect that results in a sleep-like state in humans and animals, with a greater safety margin than prior hypnotics. Some recent treatment for insomnia has also used non-benzodiazepine compounds, including zolpidem, which is a pyrazolopyrimidine-based compound.

The present invention addresses the need for improved sleep aid dosage forms, particularly those suited for treatment of elderly patients.

### SUMMARY

Orally dissolvable dosage forms comprising sleep aids such as benodiazepines are described. The dosage forms may be in the form of tablets or strips. In certain embodiments, the dosage forms may comprise particles, granules, microgranules, or crystals, and a matrix. The particles, granules or microgranules comprise a combination of immediate-release particles, granules or microgranules with sustained-release, delayed-release or enteric-coated particles, granules or microgranules. In one embodiment, the sleep aid is diazepam, triazolam, midazolam, temazepam, flurazepam, zolpidem, zaleplon or a combination thereof. The sleep aids are typically employed in pharmaceutically effective amounts to treat insomnia.

These and other embodiments, advantages and features of the present invention become clear when detailed description and examples are provided in subsequent sections.

### DETAILED DESCRIPTION

Chemical Description and Terminology

The use of the terms “a” and “an” and “the” and similar referents (especially in the context of the following claims) are to be construed to cover both the singular and the plural, unless otherwise indicated herein or clearly contradicted by context. The terms “comprising”, “having”, “including”, and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in a suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention as used herein, the terms wt %, weight percent, percent by weight, etc. are equivalent and interchangeable.

The term “active agent” is meant to include solvates (including hydrates) of the free compound or salt, crystalline and non-crystalline forms, as well as various polymorphs. Unless otherwise specified, the term “active agent” is used herein to indicate a sleep aid or a pharmaceutically acceptable salt thereof. For example, an active agent can include all optical isomers of sleep aids and all pharmaceutically acceptable salts thereof either alone or in combination.

“Pharmaceutically acceptable salts” includes derivatives of sleep aids, wherein the sleep aids are modified by making non-toxic acid or base addition salts thereof, and further refers to pharmaceutically acceptable solvates, including hydrates, of such compounds and such salts. Examples of pharmaceutically acceptable salts include, but are not limited to, mineral or organic acid addition salts of basic residues such as amines; alkali or organic addition salts of acidic residues; and the like, and combinations comprising one or more of the foregoing salts. The pharmaceutically acceptable salts include non-toxic salts and the quaternary ammonium salts of the sleep aids. For example, non-toxic acid salts include those derived from inorganic acids such as hydrochloric, hydrobromic, sulfuric, sulfamic, phosphoric, nitric and the like; other acceptable inorganic salts include metal salts such as sodium salt, potassium salt, cesium salt, and the like; and alkaline earth metal salts, such as calcium salt, magnesium salt, and the like, and combinations comprising one or more of the foregoing salts. Pharmaceutically acceptable organic salts includes salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like; organic amine salts such as triethylamine salt, pyridine salt, picoline salt, ethanolamine salt, triethanolamine salt, dicyclohexylamine salt, N,N′-dibenzylethylenediamine salt, and the like; and amino acid salts such as arginate, asparginate, glutamate, and the like; and combinations comprising one or more of the foregoing salts.

By “oral dosage form” is meant to include a unit dosage form prescribed or intended for oral administration. An oral dosage form may or may not comprise a plurality of subunits such as, for example, microcapsules or microtablets, packaged for administration in a single dose.

By “subunit” is meant to include a composition, mixture, particle, etc., that can provide an oral dosage form alone or when combined with other subunits. By “part of the same subunit” is meant to refer to a subunit comprising certain ingredients.

Dissolution profile as used herein, means a plot of the cumulative amount of active ingredient released as a function of time. The dissolution profile can be measured utilizing the Drug Release Test <724>, which incorporates standard test USP 26 (Test <711>). A profile is characterized by the test conditions selected. Thus the dissolution profile can be generated at a preselected apparatus type, shaft speed, temperature, volume, and pH of the dissolution media.

Release forms may also be characterized by their pharmacokinetic parameters. “Pharmacokinetic parameters” are parameters which describe the in vivo characteristics of the active agent over time, including for example the in vivo dissolution characteristics and plasma concentration of the active agent. By “Cₘₐₓ” is meant the measured concentration of the active agent in the plasma at the point of maximum concentration. By “C₂₄” is meant the concentration of the active agent in the plasma at about 24 hours. The term “Tₘₐₓ” refers to the time at which the concentration of the active agent in the plasma is the highest. “AUC” is the area under the curve of a graph of the concentration of the active agent (typically plasma concentration) vs. time, measured from one time to another.

By “instant-release” is meant a dosage form designed to ensure rapid dissolution of the active agent by modifying the normal crystal form of the active agent to obtain a more rapid dissolution. By “immediate-release”, it is meant a conventional or non-modified release in which greater then or equal to about 75% of the active agent is released within two hours of administration, preferably within one hour of administration.

By “controlled-release” it is meant a dosage form in which the release of the active agent is controlled or modified over a period of time. Controlled can mean, for example, sustained-, delayed- or pulsed-release at a particular time. Alternatively, controlled can mean that the release of the active agent is extended for longer than it would be in an immediate-release dosage form, e.g., at least over several hours.

Dosage forms can be combination dosage forms having both immediate release and controlled release characteristics, for example, a combination of immediate release pellets and controlled release pellets. The immediate release portion of the dosage form may be referred to as a loading dose.

Certain formulations described herein may be “coated”. The coating can be a suitable coating, such as, a functional or a non-functional coating, or multiple functional and/or non-functional coatings. By “functional coating” is meant to include a coating that modifies the release properties of the total formulation, for example, a sustained-release coating. By “non-functional coating” is meant to include a coating that is not a functional coating, for example, a cosmetic coating. A non-functional coating can have some impact on the release of the active agent due to the initial dissolution, hydration, perforation of the coating, etc., but would not be considered to be a significant deviation from the non-coated composition.

The term “rapidly disintegrating” means that the dosage form dissolves in an aqueous media within 5 minutes, specifically less than two minutes and most specifically less than one minute.

The term “orally dissolvable dosage form” refers to dosage formulations which disintegrate rapidly in the saliva of the buccal cavity and can be swallowed easily with or without drinking water.

The term “effervescent disintegration agent” includes compounds which evolve gas. Suitable effervescent agents evolve gas by means of chemical reactions which take place upon exposure of the effervescent disintegration agent to water and/or to saliva in the mouth. The bubble or gas generating reaction may be the result of the reaction of a soluble acid source and an alkali metal carbonate or carbonate source. The reaction of these two general classes of compounds produces carbon dioxide gas upon contact with water included in saliva.

Dosage Forms

Disclosed herein are dosage forms comprising sleep aids such as benzodiazepine hypnotic agents. Suitable sleep aids include benzodiazepine drugs capable of exhibiting a hypnotic activity including, for example, diazepam, triazolam, midazolam, temazepam, flurazepam or a combination thereof. Other suitable sleep aids include, for example, zolpidem, zaleplon or a combination thereof. The sleep aids are typically employed in pharmaceutically effective amounts to treat insomnia.

A problem in the treatment of patients such as the elderly and children is an inability or unwillingness to swallow solid dosage forms such as tablets. To overcome these problems, the pharmaceutical industry has developed syrups, elixirs, microcapsule containing slurries and unique tablets which dissolve in liquid prior to being consumed. Unfortunately, each of these dosage forms has its own limitations. Often, such dosage forms are more costly than traditional solid dosage forms such as simple tablets or capsules, both in terms of production, but also packaging. As an alternative, in-mouth disintegrable dosage forms for the delivery of drugs are known. Such tablets and strips may have significant advantages over other dosage forms.

In one embodiment, the in-mouth disintegrable dosage forms comprise at least one particle comprising at least one drug active and an inert excipient. In one embodiment, the dosage form exhibits a dissolution profile such that after 10 minutes at least about 87%, at least about 90%, at least about 91%, at least about 92%, at least about 93%, at least about 94%, at least about 95%, at least about 96%, at least about 97%, at least about 98%, at least about 99%, or at least about 99.5%, of the drug active exclusive from the influence of any other additives within the drug active particle. More preferably, the dosage forms exhibit a dissolution profile such that after 3 minutes at least about 87%, at least about 90%, at least about 91%, at least about 92%, at least about 93%, at least about 94%, at least about 95%, at least about 96%, at least about 97%, at least about 98%, at least about 99%, or at least about 99.5%, of the drug active exclusive from the influence of any other additives within the drug active particle. The dissolution may, for example, be measured in a USP Apparatus 1, Baskets@ 100 rpm using 900 mL media.

Orally dissolvable dosage forms, also called in-mouth disintegrable dosage forms, include, for example, chewable tablets; oral dosage forms including microparticles and effervescents which rapidly disintegrate in the mouth and provide adequate taste-masking; rapidly dissolvable, freeze-dried formulations that produce a rapidly dissolving tablet; dosage forms in which the matrix is composed of a nondirect compression filler and a lubricant, and rapidly dissolving films

In one embodiment, the orally dissolvable dosage form comprises a sleep aid in the form of a hard, compressed, rapidly disintegrating dosage form adapted for direct oral dosing. The dosage form may comprise protected particles comprising the sleep aid, and a matrix. In some embodiments, the matrix comprises a direct compression filler. In other embodiments, the matrix includes a nondirect compression filler and a lubricant, although, it may include other ingredients as well. The dosage form is adapted to rapidly dissolve in the mouth of a patient preferably in a way that provides a positive organoleptic sensation to the patient. In particular, the dosage form dissolves with a minimum of unpleasant grit which may be tactilely inconsistent with the organoleptic sensation of the dosage form.

The active agent can be provided directly, particularly when it does not have a particularly objectionable taste. The active agent may, however, be in the form of a particle, granule, microgranule or crystal protected, for example, by a protective material. This protective material can be an adsorbate, a microgranule, or a coating which forms microcapsules and/or microparticles. Combinations of these approaches are also contemplated, i.e., a coated adsorbate. In addition, protection can be provided by agglomeration or the formation of a matrix.

The protective materials for the active agent include polymers utilized in the formation of microparticles, matrix-type microparticles and microcapsules. Among these are cellulosic materials such as naturally occurring cellulose and synthetic cellulose derivatives, acrylic polymers, vinyl polymers, and copolymers comprising monomer units of one or more of the foregoing polymers. Other suitable polymers include proteinaceous materials such as gelatin, polypeptides and natural and synthetic shellacs and waxes. Protective polymers may also include ethylcellulose, methylcellulose, carboxymethyl cellulose and acrylic resin material sold under the registered trademark EUDRAGIT® by Rhone Pharma GmbH of Weiterstadt, Germany. Combinations of polymers may also be employed.

Generally, when a coating is employed, it the coating is generally used in an amount of greater than or equal to about 5 wt % based on the weight of the resulting particles, specifically greater than or equal to about 10 wt % of the weight of the particle. The upper limit of protective coating material used is generally less critical, except that where a rapid release of the active ingredient is desired, the amount of coating material should not be so great that the coating material impedes the release profile of the active agent or pharmaceutical ingredient when ingested.

When coated particles are employed, it is preferred that each individual unit of active ingredient, whether in the form of a liquid, a granule, a microgranule or a powder, be substantially completely coated. In the case of, for example, extended-release or sustained-release microparticles, (also referred to as controlled-release, delayed-release or modified-release), providing such complete coating helps ensure the desired level and type of release. When using an encapsulant or a coating to assist in taste-masking, complete coating helps ensure that the tastebuds of a patient are not exposed to the objectionable-tasting material. The more successful and complete the coating the better, in terms of its intended properties. When, for example, enteric coatings are used, substantially complete coating or encapsulation helps ensure that the active agent is not exposed to moisture or acid in the stomach. Thereafter, once the encapsulated drug reaches the intestines, the coating can, for example, disintegrate or rapidly dissolve such that it provides minimal interference with the normal dissolution profile of the drug when compared to the uncoated drug. Ingredients and methods for making particles, including microcapsules, coated granules, agglomerates, etc., are well-known in the art.

Combinations of particles, granules and microgranules having different release properties can be employed. For example, immediate-release particles, granules or microgranules can be combined with sustained-release, delayed-release or enteric-coated particles, granules or microgranules to give a dosage form having a pulsed-release profile.

Generally, the particles are provided in an amount of about 0.1 wt % to about 75 wt % based on the weight of the finished dosage form. More specifically, the particles are provided in an amount of about 1 wt % to about 60 wt %.

In one embodiment, the matrix includes at least two ingredients: a nondirect compression filler and a lubricant. Suitable nondirect compression fillers include nondirect compression sugars and sugar alcohols such as, for example, dextrose, mannitol, sorbitol, lactose, sucrose, and combinations comprising one or more of the foregoing sugars. Of course, dextrose, for example, can exist as either a direct compression sugar, i.e., a sugar which has been modified to increase its compressibility, or a nondirect compression sugar. The amount of nondirect compression filler is about 25 wt % to about 95 wt %, specifically about 50 wt % to about 95 wt % and more specifically about 60 wt % to about 95 wt %, all based on the total weight of the dosage form. Suitable hydrophobic lubricants include, for example, alkaline stearates, stearic acid, mineral and vegetable oils, glyceryl behenate, sodium stearyl fumarate, and combinations comprising one or more of the foregoing hydrophobic lubricants. Hydrophilic lubricants can also be used. The amount of lubricant is about 1 wt % to about 2.5 wt %, specifically about 1.5 wt % to about 2 wt % based on the total weight of the dosage form.

In addition to the ingredients previously discussed, the matrix may also include wicking agents, noneffervescent disintegrants and effervescent disintegrants. Wicking agents are compositions which are capable of drawing water up into the dosage form. They help transport moisture into the interior of the dosage form. In that way the dosage form can dissolve from the inside, as well as from the outside. Wicking agents include, for example, microcrystalline cellulose (AVICEL PH 200, AVICEL PH 101), Ac-Di-Sol (Croscarmelose Sodium) and PVP-XL (a crosslinked polyvinylpyrrolidone); starches and modified starches, polymers, and gum such as arabic and xanthan. Hydroxyalkyl cellulose such as hydroxymethylcellulose, hydroxypropylcellulose and hydroxyopropylmethylcellulose, as well as compounds such as carbopol may be used as well.

In another embodiment, an orally dissolvable dosage form comprises a mixture incorporating at least one water and/or saliva activated effervescent disintegration agent and particles such as microparticles. The particles incorporate a pharmaceutical ingredient together with a protective material substantially encompassing the pharmaceutical ingredient. The particles or microparticles can be substantially the same as those described previously in this application.

The effervescent disintegrating agent comprises an acid source and a carbonate source. The acid sources or acid may be any which are safe for human consumption and may generally include food acids, acid anhydrides, acid salts, and combinations comprising one or more of the foregoing acids. Food acids include citric acid, tartaric acid, malic acid, fumaric acid, adipic acid, succinic acids, and combinations comprising one or more of the foregoing food acids. Acid anhydrides of the above described acids may also be used. Acid salts include sodium, dihydrogen phosphate, disodium dihydrogen pyrophosphate, acid citrate salts, sodium acid sulfite, and combinations comprising one or more of the foregoing acid salts. Carbonate sources include dry solid carbonate and bicarbonate salts such as sodium bicarbonate, sodium carbonate, potassium bicarbonate and potassium carbonate, magnesium carbonate and sodium sesquicarbonate, sodium glycine carbonate, L-lysine carbonate, arginine carbonate, amorphous calcium carbonate, and combinations comprising one or more of the foregoing carbonate sources.

The amount of effervescent disintegration agent present in the tablet should be effective to provide an effervescent sensation in the mouth of the patient who consumes the tablet. Thus, the patient should be able to perceive a distinct sensation of “fizzing” or bubbling as the tablet disintegrates in the mouth. To provide this sensation, the amount of effervescent agent in each tablet desirably is arranged to provide about 20 to about 60 cm³ of gas. The “fizzing” sensation substantially enhances the organoleptic effects of the tablet. In general, the amount of effervescent disintegration agent useful for the formation of tablets is about 5 wt % to about 50 wt % of the final composition, and specifically about 15 wt % to about 30 wt %.

The dosage form may further include one or more additional adjuvants which can be chosen from those known in the art including flavors, diluents, colors, binders, filler, compaction vehicles, non-effervescent disintegrants, and combinations comprising one or more of the foregoing adjuvants. Examples of binders which can be used include acacia, tragacanth, gelatin, starch, cellulose materials such as methyl cellulose and sodium carboxy methyl cellulose, alginic acids and salts thereof, magnesium aluminum silicate, polyethylene glycol, guar gum, polysaccharide acids, bentonites, sugars, invert sugars, and combinations comprising one or more of the foregoing binders. Binders may be used in an amount of up to about 60 wt % of the total composition. Non-effervescent disintegrants include starches as corn starch, potato starch and modified starches thereof, sweeteners, clays, such as bentonite, micro-crystalline cellulose, alginates, gums such as agar, guar, locust bean, karaya, pecitin, tragacanth, and combinations comprising one or more of the foregoing non-effervescent distintegrants. Disintegrants may comprise up to about 20 wt % of the total weight of the composition. Coloring agents may include titanium dioxide, and dyes suitable for food such as those known as F. D. & C. dyes and natural coloring agents such as grape skin extract, beet red powder, beta-carotene, annato, carmine, turmeric, paprika, and the like. The amount of coloring used may be about 0.1 to about 3.5 wt % of the total composition.

In yet another embodiment, an orally disintegrating solid dosage form comprises a sleep aid, a filler, a binder, a taste enhancing agent, a disintegrant, and optionally a stabilizer. The dosage form may comprise about 0.1 wt % to about 20 wt %, specifically about 0.25 wt % to about 10 wt % of a sleep aid; about 40 wt % to about 95 wt %, specifically about 60 wt % to about 90 wt % of the filler, about 0.5 wt % to about 20 wt %, specifically about 1.0 wt % to about 10 wt % of the binder; about 0.5 wt % to about 15 wt %, specifically about 1.0 wt % to about 10 wt % of the taste enhancing agent; about 0.5 wt % to about 20 wt %, specifically about 1.0 wt % to about 15 wt % of the disintegrant, and 0 wt % to about 15 wt %, specifically about 0.5 wt % to about 10 wt % of a stabilizer, all based on the total weight of the final dosage formulation.

The filler used in the formulation is a pharmaceutically acceptable filler or diluent. Some suitable fillers are lactose, starch, dextrose, sucrose, fructose, maltose, mannitol, sorbitol, kaolin, microcrystalline cellulose, powdered cellulose, and combinations comprising one or more of the foregoing fillers. The filler may comprise a mixture of water soluble fillers to reduce the chance of unpleasant grittiness when the tablet dissolves in the oral cavity of the patient. Most specifically, the filler may comprise a direct compression sugar such as confectioners sugar, dextrates, dextrin, dextrose, fructose, maltose, mannitol, polydextrose, sorbitol, or other sugars and sugar derivatives.

The binder is a pharmaceutically acceptable binder. The binder is suitably a water soluble polymer such as, for example, polyvinyl alcohol, polyvinylpyrrolidone, methylcellulose, hydroxypropyl cellulose, hydroxymethyl cellulose, and combinations comprising one or more of the foregoing binders.

The disintegrant may comprise corn starch, croscarmelose sodium, crospovidone (polyplasdone XL-10), sodium starch glycolate (EXPLOTAB or PRIMOJEL), and combinations comprising one or more of the foregoing disintegrants.

The flavoring agents include taste enhancing agents and can include. artificial sweeteners such as aspartame, saccharin, dipotassium glycyrrhizinate, stevia, thaumatin and flavorants such as citric acid, peppermint oil, wintergreen oil, menthol, lemon, lime, orange grape, cherry and vanilla extract.

The stabilizers are those commonly known in the industry and the selection will depend upon the properties of the active agent employed in the dosage formulation. For example, if the active agent is sensitive to basic environments, an acidic stabilizer should be used such as citric, fumaric or tartaric acid. Similarly if the active agent is sensitive to acidic environments, a basic stabilizer should be used such as sodium dihydrogen phosphate, calcium or magnesium carbonate, arginine, lysine or meglamine. The formulations may also comprise conventional processing aids such as tablet lubricants (magnesium stearate, sodium stearate), glidants (colloidal silicon dioxide) and wetting agents or solubilizers (sodium lauryl sulfate, polysorbates). The processing aids are generally added to the dosage formulation in small amounts (less than about 5 wt % of the total weight of the formulation) and do not materially affect the properties of the final dosage formulation. Some of the aforementioned excipients can perform more than one function in the formulation.

The dosage form can be made by: a) preparing a wet granulation of the active agent, a binder, a directly compressible filler, a taste enhancing agent, a distintegrant and optionally a stabilizer; b) blending the granules from step (a) with additional filler, taste enhancing agent, disintegrants and optionally a stabilizer; and c) compressing the blend of step (b) into a tablet.

In another embodiment, the orally dissolvable dosage form is a freeze-dried dosage form that disintegrates rapidly in the mouth. To be capable of being disintegrated rapidly, the freeze-dried dosage form may comprise a network of pharmaceutically acceptable water soluble or water-dispersible carrier material. Suitable carrier materials include, for example, gelatin (including partially hydrolysed gelatin), polysaccharides such as hydrolysed dextran, dextrin and alginates (e.g. sodium alginate), and mixtures of the above mentioned carriers with each other with other carrier materials such as polyvinyl alcohol, polyvinylpyrrolidine or acacia.

Excipients which may also be employed in the freeze-dried dosage forms include preservatives, flavouring aids, colouring aids, sweeteners, fillers, and mixtures thereof. Suitable preservatives include one or more alkyl hydroxybenzoates or salts thereof, such as methyl, ethyl, propyl and/or butyl hydroxybenzoates; sorbic acid or a salt thereof; benzoic acid or a salt thereof; and mixtures thereof. Suitable flavouring aids include strawberry, cherry, mint and caramel flavouring aids, in particular strawberry flavouring aid. Suitable sweeteners include, for example, sugars such as sucrose, lactose and glucose; cyclamate and salts thereof; saccharin and salts thereof; and aspartame. Suitable fillers include polyhydric alcohols, such as mannitol, sorbitol and xylitol, or mixtures thereof, which improve the physical properties of the freeze-dried dosage form.

Conveniently an aqueous composition of the components is prepared, poured into suitable moulds, frozen, freeze-dried, and then sealed with a covering sheet adhered to the mould so as to enclose the dosage form. Preferably the dosage forms are packed in a double foil peel-back pack.

In some embodiments, the orally dissolvable dosage form is a mucoadhesive formulation such as a sheet or a strip. The dosage form may comprise, for example, an adhesive layer comprising a carboxyvinyl polymer, a water-insoluble methacrylic copolymer, a polyhydric alcohol and a sleep aid; disposed on a water-impermeable and water-insoluble carrier layer. The carboxyvinyl polymer includes polyacrylic acid, a partly crosslinked product thereof, for example, an acid type product such as Carbopol which is a commercially available product. The water-insoluble methacrylic copolymer used for the adhesive layer includes all copolymers which are usually used as a coating agent for tablets etc., for example, ethyl acrylate-methyl methacrylate-trimethylammonium-ethyl methacrylate chloride copolymer, dimethylaminoethyl methacrylate-methyl methacrylate copolymer, and the like, which may be used alone or in combination of two or more thereof. The polyhydric alcohol includes, for example, glycerin, propylene glycol, polyethylene glycol, 1,3-butanediol, sorbitol, and the like, which may be used alone or in combination of two or more thereof.

The carboxyvinyl polymer and the water-insoluble methacrylic copolymer contained in the adhesive layer are used in a ratio of 5:1 to 200:1 by weight, specifically 50:1 to 150:1 by weight (carboxyvinyl polymer:methacrylic copolymer), and the amount of both is about 40 wt % to about 98 wt %, specifically about 60 wt % to about 95 wt %, based on the total weight of all components in the adhesive layer. The polyhydric alcohol is incorporated in an amount of about 1 wt % to about 50 wt %, specifically about 5 wt % to about 20 wt %, based on the total weight of all components in the adhesive layer. The amount of the pharmaceutically active agent depends on the kinds of the agents and the desired effect thereof, but is usually about 0.1 wt % to about 50 wt %, specifically about 1 wt % to about 30 wt %, based on the total weight of the adhesive layer.

The carrier layer comprises a pharmaceutically acceptable water-insoluble, film-forming high molecular weight compound and a plasticizer. The water-insoluble, film-forming high molecular weight compound used for the carrier layer includes, for example, water-insoluble cellulose derivatives, such as ethyl cellulose having 1.5 or more of degree of substitution of ethoxy group, cellulose acetate phthalate, hydroxypropyl methyl cellulose phthalate, and the like, which may be used alone or in combination of two or more thereof. The plasticizer includes, for example, castor oil, triacetin, the same polyhydric alcohols as mentioned above, which may be used alone or in combination of two or more thereof.

The adhesive layer has a thickness of 10 to 480 μm, specifically 20 to 300 μm, and the carrier layer has a thickness of 10 to 200 μm, preferably 20 to 150 μm. The adhesive preparation comprising the adhesive layer and carrier layer has a total thickness of 20 to 500 μm, preferably 50 to 350 μm.

The components for the adhesive layer may be uniformly mixed in an appropriate solvent (e.g. ethyl alcohol, etc.), and the mixture is spread onto a release paper in a desired thickness in a conventional manner, and then it is dried to give a sheet-like adhesive layer. Separately, the components for the carrier layer may be dissolved in an appropriate solvent likewise, and the mixture is spread and laminated onto the sheet-like adhesive layer as prepared above, and then dried. The laminated sheet thus obtained is cut in a desired size to give the desired sheet-shaped adhesive preparation profile applicable to the oral cavity.

Alternatively, the sheet-like adhesive layer as prepared above is cut in a desired size, and thereon a solution of the components for the carrier layer is sprayed or coated so that the carrier layer covers and surrounds the adhesive layer, and dried to give the desired sheet-shaped adhesive preparation profile.

In another embodiment, a mucoadhesive formulation comprises an anhydrous but hydratable mucoadhesive monolithic polymeric matrix which includes amorphous fumed silica present in an amount sufficient to enhance adhesion of the polymeric matrix to mucosal tissue. Optionally, the matrix may include a plasticized, film-forming cellulose ester. Suitable polymers for the polymeric matrix are polyols, e.g., polyethylene glycols (PEG) having a number average molecular weight in the range of about 1500 to about 8500, preferably in the range of about 4000 to about 8000. These polyols promote lowering of the dielectric constant of the aqueous environment to which the carrier is exposed and solubilize relatively insoluble agents thereby increasing the thermodynamic activity of the therapeutic agent in the monolithic polymer matrix. Amorphous fumed silica is added to the polymeric matrix in a processable dry powder form. The amount of fumed silica present varies, depending on the nature of the polymeric matrix utilized in any given instance; however, the amount of fumed silica added is sufficient to enhance the adhesion of the polymeric matrix to mucosal tissue. If the polymeric matrix is constituted primarily by a polyol, the amount of fumed silica present preferably is at least about 10 percent by weight of the matrix. On the other hand, if the polymeric matrix also includes other known bioadhesives such as a carboxy-functional polymer, the amount of fumed silica present can be as low as about 2 percent by weight of the matrix, and preferably is at least about 3 percent by weight of the matrix. An optional bioadhesive such as a water-swellable, but water-insoluble, fibrous, cross-linked carboxy-functional polymer can also be present. Other optional bioadhesives include polymers which are hydrophilic and water-dispersible, have free carboxylic groups and a relatively high base binding capacity.

The resulting monolithic polymer matrix can be optionally laminated to a water-insoluble, but water-permeable or substantially water-impermeable, barrier film which does not contain the active agent. The barrier film serves as a backing to prevent or minimize back diffusion of the active agent and protect the extraneous tissues, such as gums and teeth, from direct exposure to the matrix. The barrier film may include pharmaceutically edible adjuvants. Illustrative adjuvants include hydrogenated vegetable oil, microcrystalline cellulose, methylcellulose, calcium phosphate dihydrate, talc, kaolin, bentonite, hydroxypropyl cellulose, high melting glyceryl esters such as glyceryl behenate, methylcellulose, cellulose acetate butyrate, polyvinyl pyrrolidone, polyvinyl alcohol, magnesium stearate, silicon dioxide, and stearic acid.

In yet another embodiment, a mucoadhesive composition comprises a water-soluble polymer, a polyalcohol, and an active agents. Optionally, the formulation may contain a combination of certain plasticizers or surfactants, colorants, sweetening agents, flavors, flavor enhancers, or other excipients commonly used to modify the taste of formulations intended for application to the oral cavity. The resulting film in characterized by an instant wettability which causes the film to soften immediately after application to the mucosal tissue thus preventing the patient from experiencing any prolonged adverse feeling in the mouth, and a tensile strength suitable for normal coating, cutting, slitting, and packaging operations.

The polymers used for the mucoadhesive film include polymers which are hydrophilic and/or water-dispersible. Suitable polymers include water-soluble cellulose-derivatives. Hydroxypropylmethyl cellulose, hydroxyethyl cellulose, or hydroxypropyl cellulose, either alone, or mixtures thereof, are particularly suitable. Other optional polymers include polyvinyl pyrrolidone, carboxymethyl cellulose, polyvinyl alcohol, sodium alginate, polyethylene glycol, natural gums like xanthane gum, tragacantha, guar gum, acacia gum, arabic gum, water-dispersible polyacrylates like polyacrylic acid, methylmethacrylate copolymer, carboxyvinyl copolymers. The concentration of the water-soluble polymer in the final film can vary between 20 and 75 wt %, preferably between 50 and 75 wt %.

The polyalcohol is used to achieve the desired level of glycerol, polyethylene glycol, propylene glycol, glycerol monoesters with fatty acids or other pharmaceutically used polyalcohols. The concentration of the polyalcohol in the dry film usually ranges between 0.1 and 5 wt %.

The surfactants used for the mucoadhesive film may be one or more nonionic surfactants. When a combination of surfactants is used, the first component may be a polyoxyethylene sorbitan fatty acid ester or a α-hydro-ω-hydroxypoly (oxyethylene)poly(oxypropylene)poly(oxyethylene) block copolymer, while the second component may be a polyoxyethylene alkyl ether or a polyoxyethylene castor oil derivative. Specifically, the HLB value of the polyoxyethylene sorbitan fatty acid ester should be between 10 and 20, whereby a range of 13 to 17 is particularly preferred. The α-hydro-ω-hydroxypoly(oxyethylene)poly(oxypropylene)poly(oxyethylene) block copolymer should contain at least 35 oxypropylene-units, preferably not less than 50 oxypropylene-units.

In some embodiments, the orally dissolvable dosage form enables first of all a sufficient blood level of sleep aid to be obtained rapidly after administration in order to induce sleep, and then a second pulse of sleep aid to be released after a fixed time after administration in order to maintain sleep. The dosage form may thus comprise two release pulses, the first being immediate and the second being delayed to a fixed time. The immediate release pulse can liberate about 40 to about 70% of the sleep aid. The delayed release should be completed at a time after administration compatible with the desired time of sleep, and the time needed for elimination of the drug from the human body to a sufficiently low level roughly 8 hours after administration. In view of this, the time to initiate the release of the delayed-release pulse is about 1 to about 4 hours after administration of the dosage form.

Dissolution may, for example, be measured in a USP Apparatus 1, Baskets @ 100 rpm, Drug Release Test 1 using 900 mL of pH 1.2 buffer for 1.5 hours followed by testing in 900 mL of pH 6.8 buffer. Alternatively, dissolution may be measured at a single pH such as pH 6.8. The dissolution profile of such a dosage form may be, for example,

30 wt % to 54 wt % of the sleep aid released by 10 minutes; and

40 wt % to 70 wt % of the sleep aid released by 30 minutes; and

30 wt % to 60 wt % of the sleep aid released between 1 and 4 hours.

In general, the amount of active ingredient incorporated in each tablet or dosage form may be selected according to known principles of pharmacy. An effective amount of pharmaceutical ingredient is specifically contemplated. By the term “effective amount”, it is understood that, with respect, to for example, pharmaceuticals, a “pharmaceutically effective amount” is contemplated. A “pharmaceutically effective amount” is the amount or quantity of an active agent which is sufficient to elicit the required or desired therapeutic response, or in other words, the amount which is sufficient to elicit an appreciable biological response when administered to a patient. In one embodiment, the amount of active ingredient incorporated in each tablet or dosage form can be from about 1 mg to about 50 mg, and more preferably, from 7.5 mg to 30 mg.

### EXAMPLE 1

### Formulation of a First Dosage Form

Sleep aid formulation using a non-direct compression sugar:

| | |
|---|---|
| Coated Sleep Aid Powder (78.2%) | 15.7% |
| Powdered Mannitol (non-direct | 64.0% |
| compression sugar), USP | |
| Sodium Bicarbonate, No. 1 USP | 2.3% |
| Citric Acid, Anhydrous Fine | 1.7% |
| Granular USP | |
| Artificial sweetener, Dried | 4.6% |
| Wicking Agent | 5.8% |
| Glidant | 0.3% |
| Magnesium Stearate, NF | 1.5% |
| Artificial Flavor | 3.8% |
| Artificial Color | 0.3% |
| | 650.0 mg Total |
| | tablet weight |

Tablets are produced using a direct compression method as follows: All of the material, except the lubricant are weighed and blended for a period of about 30 to about 50 minutes. Thereafter, the lubricant is added and the mixture is blended for an additional 5 to 15 minutes. The blend is then tableted on a conventional 6 or 16 stage rotating tablet press at 25-30 revolutions per minute. Tablets are compressed using an average compression force of 10.36 kN—the average ejection force is 184.6 N. The result is a fast dissolving tablet with a minimum of grit and a pleasant organoleptic experience.

### EXAMPLE 2

### Formulation of a Second Dosage Form

Microparticles comprising a sleep aid are tableted into an effervescent tablet of about 1.0-2.0 kilo pounds hardness with an effervescent disintegration agent and other ingredients according to the following recipe:

| Ingredient | Mg/Tablet |
|---|---|
| Mannitol | 225.0 mg |
| Aspartame | 40.0 mg |
| Cherry Flavor | 6.0 mg |
| Magnesium Stearate | 5.0 mg |
| Silicon Dioxide | 1.0 mg |
| Sodium Bicarbonate | 100.0 mg |
| Citric Acid | 80.0 mg |
| Microparticles | 94.3 mg |

The effervescent tablet has a dissolution time of less than about 1.0 minutes.

### EXAMPLE 3

### Formulation of a Third Dosage Form

A mucoadhesive dosage form is formulated as follows:

Composition for Adhesive Layer:

| | | |
|---|---|---|
| Hiviswako (carboxyvinyl polymer) | 12 | g |
| Eudragit RS | 0.12 | g |
| Polyethylene glycol 400 | 2 | g |
| Titanium oxide | 0.6 | g |
| Sleep aid | 0.06 | g |
| Ethanol | 140 | ml |

The above ingredients are mixed and dissolved by kneading to give a uniform paste.

Composition for Carrier Layer:

| | | |
|---|---|---|
| Ethocel (STD) (ethyl cellulose having ethoxy | 15 | g |
| content of 48-49.5) | | |
| Castor oil | 4 | g |
| Red No. 2 pigment | 10 | mg |
| Ethanol | 140 | ml |

The above ingredients are mixed well to give a uniform paste. The composition for adhesive layer prepared above is spread onto a release paper and dried to give a sheetlike adhesive layer (thickness of the adhesive layer: 100 μm). Subsequently, the composition for carrier layer is spread onto the sheet-like adhesive layer and then dried to form a carrier layer (thickness of the carrier layer: 50 μm). The sheet thus obtained is cut in a fixed size (0.5 cm²) to give a sheet-shaped adhesive preparation profile for the oral cavity, wherein the sleep aid is contained in an amount of 25 μg per one sheet).

Embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A dosage form comprising, a sleep aid in the form of an orally dissolvable composition.

2. The composition of claim 1, wherein the dosage form is in the form of a tablet or a strip.

3. The dosage form of claim 1 or 2 wherein the sleep aid is a benzodiazepine or a non-benzodiazepine or a combination thereof.

4. The dosage form of claim 3, wherein the benzodiazepine is diazepam, triazolam, midazolam, temazepam, flurazepam or a combination thereof.

5. The dosage form of claim 3, wherein the benzodiazepine is zolpidem, or zaleplon or a combination thereof.

6. The dosage form of claim 4, wherein the benzodiazepine is present in the dosage form in an amount of about 1 mg to about 50 mg.

7. The dosage form of claim 1, wherein the sleep aid is present in the dosage form in the form of a particle, granule, microgranule or crystal comprising a protective material.

8. The dosage form of claim 6, wherein the protective material is a naturally occurring cellulose, a synthetic cellulose, an acrylic polymer, a vinyl polymer, copolymers comprising monomer units of one or more of the foregoing polymers, and combinations comprising one or more of the foregoing polymers.

9. The dosage form of claim 6, wherein the particles, granules or microgranules comprise a combination of immediate-release particles, granules or microgranules with sustained-release, delayed-release, or enteric-coated particles, granules or microgranules.

10. The dosage form of claim 6, comprising a matrix, wherein the matrix comprises a nondirect compression filler and a lubricant.

11. The dosage form of claim 6, comprising an effervescent disintegrating agent.

12. The dosage form of claim 10, wherein the effervescent disintegrating agent comprises an acid source and a carbonate source.

13. The dosage form of claim 1, comprising a filler, a binder, a taste enhancing agent, a disintegrant, and optionally a stabilizer.

14. The dosage form of claim 1, wherein the dosage form is a freeze-dried dosage form.

15. The dosage form of claim 13, comprising a carrier material, wherein the carrier material is a gelatin, a polysaccharides, an alginates, or a combination comprising one or more of the foregoing carrier materials.

16. The dosage form of claim 1, wherein the dosage form is a mucoadhesive composition.

17. The dosage form of claim 15, comprising an adhesive layer comprising a carboxyvinyl polymer, a water-insoluble methacrylic copolymer, a polyhydric alcohol and the sleep aid; disposed on a water-impermeable and water-insoluble carrier layer.

18. The dosage form of claim 15, wherein the mucoadhesive composition comprises an anhydrous but hydratable mucoadhesive polymeric matrix comprising a polymer and amorphous fumed silica present in an amount sufficient to enhance adhesion of the polymeric matrix to mucosal tissue.

19. The dosage form of claim 15, wherein the mucoadhesive composition comprises a water-soluble cellulose-derivative polymer, and a polyalcohol.

20. The dosage form of claim 1, wherein the dosage form comprises an immediate-release pulse of sleep aid and a delayed-release pulse of sleep aid.

21. The dosage form of claim 19, wherein the dissolution profile measured in a USP Apparatus 1, Baskets@ 100 rpm, Drug Release Test 1 using 900 mL is:
30 wt % to 54 wt % of the sleep aid released by 10 minutes; and
40 wt % to 70 wt % of the sleep aid released by 30 minutes; and
30 wt % to 60 wt % of the sleep aid released between 1 and 4 hours.
